(19) United States
(12) Patent Application Publication
(10) Pub. No.: US 2026/0240632 A1
(43) Pub. Date: Aug. 20, 2026

(54) A DETACHABLE SLEEVE FOR A HANDHELD INTRAORAL SCANNER AND A METHOD FOR MANUFACTURING SUCH A SLEEVE

(71) Applicant: 3SHAPE A/S, Copenhagen K (DK)

(72) Inventors: Anders LARSEN, Copenhagen K (DK); Christoph HANSEN, Copenhagen K (DK)

(73) Assignee: 3SHAPE A/S, Copenhagen K (DK)

(21) Appl. No.: 19/489,629

(22) PCT Filed: Jun. 7, 2024
(86) PCT No.: PCT/EP2024/065787

§ 371 Date: Dec. 3, 2025

(30) Foreign Application Priority Data
Jun. 9, 2023 (DK) PA202370279

(51) Int. Cl. A61C 9/00

(52) U.S. Cl. CPC A61C 9/0053

(57) ABSTRACT

A handheld intraoral scanner system includes a handheld intraoral scanner and a detachable sleeve configured to be detachably attached to a scan tip of the scanner. The sleeve includes a cover part and a window part, which includes a tapered portion. The window part may be injection molded. The sleeve may be formed by two-component injection molding the cover part and the window part. The window part includes an optical area having a lower birefringence than a relief area located at the tapered portion and including an injection site at which a first molten polymer material was injected into a first form, forming the window part. A method of injection molding a window part for a detachable sleeve that includes a cover part and that is configured to be detachably attached to a handheld intraoral scanner. The method may include two-component injection molding the cover part and the window part.

### FIELD

The disclosure relates to a detachable sleeve for a handheld intraoral scanner and a method for manufacturing a such detachable sleeve. In particular, the disclosure relates to a detachable sleeve having improved optical characteristics for a handheld intraoral scanner and a method for manufacturing a such detachable sleeve having improved optical characteristics.

### BACKGROUND

When scanning teeth of a patient using a handheld intraoral scanner, it is important that the equipment used meet high hygienic standards, in particular parts that come in contact with the patient's mouth, such as the scan tip. This is to ensure that no cross-contamination occurs between patients, and to ensure that the patient does not come in contact with remains of body liquids (e.g. saliva and blood) that may remain on parts of the handheld intraoral scanner from a previous scan, and risking getting potentially infected by bacteria or viruses. For this purpose, autoclavable scan tips are applied to cover scan heads of handheld intraoral scanners, that are suitable for being cleaned and disinfected using an autoclave after use, in order to be reused.

However, under some conditions, it is environmentally more appropriate to use single-use scan tips than using autoclavable scan tips, since autoclaving an autoclavable scan tip through out it's lifetime may lead to more CO2-emissions than producing, using, and disposing single-use scan tips during the same amount of time, as the autoclavable scan tip's lifetime.

Operating a handheld intraoral scanner with single-use scan tips requires a large amount of single-use scan tips and thus a high production rate of such scan tips. To meet the high production rate, single-use scan tips may be manufactured by injection molding, which allows for high production rates of products.

For a handheld intraoral scanner to work properly, polarized light may be emitted from the handheld intraoral scanner and reflected back into the handheld intraoral scanner after reflecting from a dental object (e.g. a tooth). When the scan head of the handheld intraoral scanner is covered by a protective scan tip, the scan tip needs to have a transparent part, such as a window, for allowing the polarized light to pass. However, when the window of the scan tip is manufactured in high rates by injection molding, the window may comprise high amount of stress, which may lead to a high amount of birefringence. Birefringence is a property of some materials to refract a beam of light into two different directions. Thus, high amounts of birefringence may lead to an increase in refraction of the polarized light that is emitted from the handheld intraoral scanner and returning back to the handheld intraoral scanner, and may thus lead to reduce the quality of the intraoral scan.

Birefringence is usually expressed as the difference between the refractive indices of the two orthogonal polarization directions in a material, and it can provide information about the structure, composition, and physical properties of the material. Conventionally it is denoted in units of [nm/cm], to illustrate the size of the effect per unit thickness of the media. Retardation, in the context of birefringence, refers to the difference in the path length [nm] that the two light waves of different polarization take through a birefringent material in different directions.

Forming the windows in industrial ovens is too time consuming, the number of parts are limited due to the space inside the industrial oven, and require more time for the parts to cool down.

### SUMMARY

An aspect of the present disclosure is to provide a detachable sleeve comprising a window that allows for a reduction of refraction of polarized light when passing through the window.

A further aspect of the present disclosure is to provide a method for allowing a higher production rate of detachable sleeves comprising a window that allows for a reduction of refraction of polarized light when passing through the window.

According to the aspect, a detachable sleeve that may be configured to detachably cover at least a part of a handheld intraoral scanner is provided.

The detachable sleeve may be a rigid part or a flexible part. The detachable sleeve may be a part that may be slid, clamped, screwed, or in any other way fastened into position, on the handheld intraoral scanner. The detachable sleeve may be detachably (i.e. removably) attached to the handheld intraoral scanner, such that the detachable sleeve may be removed from the handheld intraoral scanner, without the use of tools.

The handheld intraoral scanner may be a handheld scanner device that is configured to acquire light information reflected from a three-dimensional dental object during a scanning session, wherein the scanning session is a period of time in which a scan is performed using the handheld intraoral scanner. The light information may be intraoral scan data and may be configured to be used to generate or update a virtual 3D model of a dental object. The dental object may be a tooth, a part of a tooth, teeth, an upper or lower jaw or parts of them, a whole dentition or a part of a dentition, and/or a gingiva or part thereof. The handheld intraoral scanner may comprise a scan tip that may be arranged at a distal end of the handheld intraoral scanner, and that may be configured to be at least partly inserted inside the oral cavity (i.e. mouth) of a patient. The scan tip may comprise an open channel that may be configured to allow light to pass through two openings each located at opposite ends of the scan tip. The scan tip at the distal end may further comprise one of the two openings. The opening at the distal end of the scan tip may be covered by a cover, such as a window, that may be made of a transparent material, such as glass or a plastic material.

The detachable sleeve may be configured to cover at least a part of the scan tip. The detachable sleeve may be detachably arranged, fastened, or attached onto the scan tip of the handheld intraoral scanner.

A detachable sleeve that is configured to be removably attached to the scan tip of a handheld intraoral scanner may be beneficial, since a such detachable sleeve may improve hygiene of the equipment, the scanning process, and the patient, since a such detachable sleeve may protect the scan tip from being contaminated by coming into contact with body liquids from the patient, such as saliva and blood, thereby protecting the equipment and a next patient. The detachable sleeve may further reduce negative impact on the environment, since using single-use sleeves such as the detachable sleeve, produces less CO2 than autoclaving multiple-use sleeves, when measured during the same time period.

The detachable sleeve may comprise a cover part and a window part. The window part may be configured to be at least partly aligned with the window of the scan tip, when the detachable sleeve is arranged into position on the scan tip, and may be configured to allow light to pass to and from the window of the scan tip.

The detachable sleeve may comprise an opening at one end that is arranged opposite to a distal end of the detachable sleeve at which the window part may be arranged. The opening may thus be arranged at one end of the cover part of the detachable sleeve. The opening of the detachable sleeve may be configured to receive at least a part of the scan tip of the handheld intraoral scanner, thereby allowing the detachable sleeve to cover the least part of the scan tip of the handheld intraoral scanner.

The window part may include a first end and a second end. The first end may be arranged at a distal end of the window part, and the second end may be arranged at a proximal end of the window part, that is opposite to the first end.

The window part may be formed by injection molding. Forming the window part by injection molding may allow for a higher production rate, than forming the window using other manufacturing methods, such as forming the window in an industrial oven.

The window part may include a tapered portion at the second end. The tapered portion may be an extension of the second end of the window part that points or tapers away from the window part. The window part may be arranged in the detachable sleeve such, that the tapered portion tapers or points towards the opening of the detachable sleeve that may be configured to receive the at least part of the scan tip of the handheld intraoral scanner. Thus, when the detachable sleeve is attached to the scan tip of the handheld intraoral scanner and thereby covering the at least part of the scan tip of the handheld intraoral scanner, the tapered portion may taper or point away from the distal end of the scan tip of the handheld intraoral scanner. The first end of the window part may thus be arranged closer to the distal end of the scan tip of the handheld intraoral scanner than the second end of the window part is. The second end of the window part may, when attached to the scan tip of the handheld intraoral scanner, be arranged closer to a user interface (e.g. physical or digital buttons) arranged on the handheld intraoral scanner, than the first end of the window part is.

Injection molding parts may imply injecting molten polymer material under high pressures, such as 800 bar and 1200 bar. Such high pressures may result in the formation of mechanical stress inside the material, which in turn may lead to an increase in birefringence in the material. The highest stress and highest birefringence in the injection molded part may be located closest to the injection site, which is the location in the injection molted part where the molten polymer material has entered the form that was used to manufacture the injection molted part.

The window part comprising the tapered portion may allow the injection site to be arranged at the tapered portion, and may thus allow the highest stress, and thus the highest birefringence, to be located and formed in the tapered portion, thereby relieving the rest of the window part from forming or containing the same level of stress and birefringence. The even increase in width of the tapered portion from the injection site towards the first end of the window part allows for a more uniform and laminar flow of a molten polymer material from the injection site during injection, that allows for a reduction of mechanical stress, and thereby also reduction of birefringence, forming at an area of the window part that is outside the tapered portion and that is closer to the first end of the window part than the tapered portion is, than if the width was constant through the entire length of the window part, such as in a substantially rectangular window. Furthermore, the distance from the injection site to the area of the window part outside the tapered portion allows for a further reduction of mechanical stress and birefringence at that area, such that the larger the distance, the less mechanical stress and birefringence is formed at the area of the window part outside the tapered portion. The detachable sleeve may be configured to be releasably attached to the handheld intraoral scanner such, that the polarized light to and from the handheld intraoral scanner only passes through the area of the window part that is outside the tapered portion and closer to the first end of the window part than to the second end of the window part, which is the area of the window part than comprises least mechanical stress and least birefringence, since polarized light through the window part may be refracted more in the tapered portion than in the rest of the window part that is closer to the first end of the window part than to the tapered portion, thereby allowing for an increase in scan quality.

Furthermore, since a window part comprising a tapered portion comprising the injection site allows for a more rapid reduction in mechanical stress and birefringence through the entire length of the window part in a direction away from the injection site and the tapered portion than in a window part not comprising a tapered portion comprising the injection site (e.g. a substantially rectangular or oval window), the increased reduction in mechanical stress and birefringence allows for a window part that is shorter in length between the first end and the second end, than a window part not comprising a tapered portion comprising the injection site, thereby allowing for a reduction in a length of the window part, thereby allowing for a reduction in material cost.

Since an area of the window part located at the first end, may be the area of the window part that is configured to be aligned with the opening of the scan tip, and may thus be the area of the window part that is configured to allow the light to pass to and from the handheld intraoral scanner. The area of the window part that may be configured to allow light to pass from and to the scan tip may be referred to as the “optical area”, which may be a field-of-view of the handheld intraoral scanner, which defines the area of the image data that is captured by each scan. Reducing the level of mechanical stress and birefringence within the optical area may be beneficial, since the polarized light to and from the scan tip of the handheld intraoral scanner may pass through the optical area of the window part of the detachable sleeve, and the lower the level of mechanical stress and birefringence is in the optical area, the less the polarized light may refract when passing through the optical area of the window part of the detachable sleeve.

Since the window part may comprise a tapered portion, the window part may comprise a width that smoothly increases in the tapered portion in a direction from the second end towards the first end. The smooth increase of the width in the tapered portion towards the first end of the window part may further contribute to a more rapid reduction of mechanical stress and a more rapid reduction of birefringence in the optical area of the window part, than in a window part of similar size without a smooth increase of width of a tapered portion. The tapered portion may thus extend flush from the window part. By extending flush from the window part, the tapered portion may not comprise sharp corners or sudden bends that may lead to the formation of mechanical stress during or after manufacturing the window part by injection molding the polymer material into the form, thereby reducing the formation of mechanical stress in the window part, thereby reducing birefringence in the window part.

Using the tapered portion as a relief area may relieve the formation of mechanical stress in the optical area, thereby reducing stress-induced birefringence in the optical area of the window part. For the same reason (i.e. reducing mechanical stress and birefringence), the tapered portion may not comprise concave corners. Concave corners are corners that extend inwardly towards the window part, thereby forming areas in the window part, wherein the molten polymer material will follow a rapidly expanding area in the form, during injection, which may lead to higher mechanical stress, and may thus result in higher levels of birefringence.

According to the aspect, a detachable sleeve configured to detachably cover at least a part of a handheld intraoral scanner is disclosed. The detachable sleeve may comprise a cover part, and a window part including a first end and a second end, wherein the window part may be formed by injection molding, and wherein the window part may include a tapered portion at the second end.

In one aspect, the cover part and the window part may be manufactured separately. For example, the cover part may be injection molded, and the window part may be injection molded in a separate molding process. The cover part and the window part may subsequently be assembled, for example by gluing the cover part and the window part together.

In another aspect, the detachable sleeve may be manufactured in one piece. The cover part and the window part may be combined based on two-component injection molding.

The detachable sleeve may for example be manufactured by injecting a first molten polymer material (e.g. Acrylonitrile Butadiene Styrene (ABS) material) into a first form, that may be shaped as the cover part of the detachable sleeve. The first form may comprise a first core for preventing the first molten material to enter an opening in the formed cover part, where the window part will subsequently be located. In a subsequent step, the first core may be substituted with a second core that allows a second molten polymer material to enter the opening where the window part will be located. In another aspect of the subsequent step, the first core may be moved such that the opening where the window part will be located is uncovered, allowing a second molten polymer material to enter the opening where the window part will be located. A second molten polymer material (e.g. Poly-methyl methacrylate material) may subsequently be injected into the opening where the window part will be located. The opening may be limited by sidewalls of the formed cover part and the substituted second core or the moved first core, thus defining a second form. The second form may thus be defined by the opening in the formed cover part and the first core that has been moved or a second core substituting the first core. The opening and the substituted second core or the moved first core may be shaped as the window part that is to be formed. The second molten polymer material may be injected at a location where the tapered portion will be formed. The second molten polymer may be injected into the second form with a pressure between 850 bar and 1150 bar. The duration of the injection may be between 150 milliseconds and 350 milliseconds.

A second pressure may subsequently be applied to the second form that may be between 850 bar and 1150 bar that may be maintained for a time period of between 350 milliseconds and 650 milliseconds.

Applying a second pressure subsequently to injecting the second molten polymer material, may allow reducing mechanical stress and resulting birefringence in the formed window part.

In another example, the detachable sleeve may be manufactured by injecting the second molten polymer material (e.g. Poly-methyl methacrylate material) into a second form shaped as the window part. The second molten polymer material may be injected at a location where the tapered portion will be formed. The second molten polymer may be injected into the second form with a pressure between 950 bar and 1050 bar. The duration of the injection may be between 210 milliseconds and 270 milliseconds.

A second pressure may subsequently be applied to the second form that may be between 850 bar and 950 bar that may be maintained for a time period of between 450 milliseconds and 550 milliseconds.

Subsequently, a first molten polymer material (e.g. Acrylonitrile Butadiene Styrene material) may be injected into a first form. The first form may be shaped as the cover part of the detachable sleeve. The first molten polymer material may be injected into the first form while the formed window part is arranged in the first form. The first form may comprise the window part while the first molten polymer material is injected into the first form, for preventing the first molten polymer material to enter a space inside the first form that is covered by the formed window part.

As described in the above examples, the detachable sleeve may be manufactured by two-component injection molding the cover part on top of the formed window part or vice versa, i.e. two-component injection molding the window part on top of the formed cover part.

Thus, two-component injection molding the cover part and the window part may occur by injecting a second molten polymer material into a second form for the cover part while the window part may be arranged within the second form, or by injecting a second molten polymer material into a second form for the cover part prior to injecting the first molten polymer material into the first form for the window part while the first form may be arranged within the cover part. For example, by injecting a second molten polymer material into a second form for the cover part and subsequently injecting the first molten polymer material into the first form for the window part while the first form may be arranged within the cover part.

Two-component injection molding the detachable sleeve may reduce the production time since an assembly step, wherein the cover parts and the window parts are subsequently assembled in a separate manufacturing process, may be omitted, and may thus allow for a lower production time and a higher production rate.

Manufacturing the detachable sleeve by combining the cover part and the window part based on two-component injection molding may allow for a faster and more efficient production of detachable sleeves, since no further processing step may be required to assemble the cover part and the window part together, since one of the cover part or the window part, may be injection molded on top of the other one, and may thereby allow for gluing the cover part and window part together during one of the first or second injection molding process. The gluing may occur due to the heat generated during injection molding.

The window part may be at least partly made of a Poly-methyl methacrylate material, a Polycarbonate material, or a Cyclic Block Copolymer (e.g. poly(cyclohexylethylene) and ethylene-co-1-butene, with an adjusted ratio). The cover part may be at least partly made of an Acrylonitrile Butadiene Styrene material, a Polycarbonate material, a Polybutylene terephthalate material, an Acrylonitrile butadiene styrene material, a Polypropylene material, a Polyethylene material, a Polyoxymethylene material, a Polystyrene material, or a Styrene-acrylonitrile resin material. The window part and the cover part may at least partly be made of a Poly-methyl methacrylate material, a Polycarbonate material, or a Cyclic Block Copolymer (e.g. poly(cyclohexylethylene) and ethylene-co-1-butene, with an adjusted ratio).

Poly-methyl methacrylate (PMMA) material is in particular suitable to be used as a window material, since injection molding Poly-methyl methacrylate using the above mentioned pressure ranges and pressure holding times may allow forming a window part with reduced levels of mechanical stress in the window part and reduced levels of birefringence.

The tapered portion may comprise rounded corners. The tapered portion may be arranged such that it extends and narrows towards the second end and may comprise a rounded corner or a blunt portion that may comprise two rounder corners. The tapered portion may comprise rounded corners where the tapered portion meets with the rest of the window part. The rounded corners may allow for a reduction in mechanical stress when formed, and thereby, a reduction in birefringence in the formed window part is obtained as no sharp corners are present in a flow path from the injection site towards the first end of the window part.

The tapered portion may be at least partly shaped as one of a trapezoid, a trapezoid having a rounded end, a trapezoid having rounded corners at the second end, a triangle, a triangle having a rounded corner at the second end, semioval, a semioval tapered towards the second end, a part of a semioval, a semicircle, or a part of a semicircle.

The geometry of the window part may have an effect on the birefringence of the window part, since sharp edges, concave corners, and complex geometry, may increase mechanical stress in the window part, which may increase the birefringence in the window part. Therefore, having a tapered portion in the window part that increases smooth in width from the second end towards the first end as the shapes described above, may allow for a more rapid reduction in birefringence in the optical area of the window part.

The window may be injection molded from an injection site. The tapered portion of the window part may comprise the injection site.

The injection site may be at a location at which the second molten polymer material may be injected into the second form. The highest mechanical stress and the highest birefringence in the window material may be located at the injection site. Therefore, arranging the injection site at the second end of the window part, and thus distantly from the first end of the window, at which the optical area of the window part is located, may relieve the optical area from increased mechanical stress, and may reduce mechanical stress, and thereby also reduce birefringence, in the optical area of the window part. Therefore, the injection site may be located at the tapered portion, and may preferably be located at the second end of the window part within the tapered portion.

As previously mentioned, the window part may comprise an optical area that may be a part of the window part. The optical area may be configured to allow light to pass from and to the scan tip. Therefore, the optical area may preferably be the part of the window part that has the lowest levels of birefringence.

The optical area may be arranged closer to the first end of the window part than to the second end of the window part. Arranging the optical area closer to the first end of the window part than to the second end of the window part may reduce the level of birefringence in the optical area since the highest levels of birefringence may be located closest to the injection site, which may be located at the second end of the window part. This may be because the tapered portion allows for a more rapid decrease of a level of birefringence in a direction from the injection site towards the first end of the window part, and therefore, a minimal birefringence is achieved at the optical area, as the optical area is arranged furthest away from the injection site. The optical area may comprise levels of birefringence between 50 and 350 nm/cm, such as for example between 100 nm/cm and 300 nm/cm. The optical area of the window part may be defined as an area, region, or part of the window part, that has a level of birefringence between 100 nm/cm and 300 nm/cm.

Reducing the level of mechanical stress and birefringence within the optical area may improve the quality of intraoral scanning since the polarized light to and from the scan tip of the handheld intraoral scanner performing the scanning, may pass through the optical area of the window part of the detachable sleeve covering the scan tip. The lower the level of mechanical stress and birefringence is in the optical area, the less the polarized light may refract when passing through the optical area of the window part of the detachable sleeve, and thus the higher the quality of the intraoral scanning may be.

The window part may comprise a relief area arranged at the second end of the window part. The relief area may be a part of the window part that is configured to contain the highest levels of mechanical stress and the highest level of birefringence, thereby relieving the optical area of the window part from containing the highest levels of mechanical stress and the highest levels of birefringence. Concentrating the highest levels of mechanical stress and the highest levels of birefringence in the relief area may be achieved by arranging the injection site within the relief area. For example, the injection site or the relief area may be arranged as far from the optical areas as possible. Arranging the injection site as far as possible from the optical area may be achieved by injecting the second molten polymer material at a location at the second form where the second end of the window part will be located. The relief area may be within the tapered portion of the window part, and may be an area of the tapered portion at which the injection site is located.

Concentrating the highest levels of mechanical stress and highest levels of birefringence in the relief area may allow the optical area to have reduced levels of mechanical stress and reduced levels of birefringence, thereby improving the quality of the intraoral scanning.

For example, the window part may have a length of 300 mm from the first end to the second end, the optical area may be located between the first end of the window part and a distance of 150 mm from the first end. The relief area may be located between the second end of the window part and 150 mm from the second end. The window part may be divided between the optical area and the relief area.

A transition area may be located between the optical area and the relief area of the window part. The transition area may be a part of the window part that comprises levels of mechanical stress and levels of birefringence that are higher than levels of mechanical stress and levels of birefringence of the optical area, but lower than the levels of mechanical stress and levels of birefringence of the relief area. The optical area may be located between the first end of the window part and the transition area. The relief area may be located between the second end of the window part and the transition area.

A ratio between a first minimum distance between the injection site and the optical area and a second minimum distance between the injection site and the first end of the window part may be between 25% and 45%. The ratio may be between 33% and 43%. The ratio may be between 36% and 40%.

The first minimum distance may be a first distance between the injection site and the closest part of the optical area to the injection site.

The second minimum distance may be a second distance between the injection site and the closest part of the first end of the window part.

Having a ratio of distances of the injection site relative to the optical area and a distance between the injection site and the distal end of the window part that are within the above mentioned limits (i.e. the above mentioned particular limit of ratio), allows the second molten polymer material to flow from the injection site towards the distal end (i.e. first end) of the window part, in a process, where less mechanical stress will propagate towards the first end of the window part, resulting in the formation of a part of the window part, that comprises levels of mechanical stress that result in levels of birefringence, that are below 300 nm/cm, thereby resulting in the formation of the above described optical area, that allows for an improved quality in intraoral scanning.

For example, the injection site may be located at a distance between 1 mm and 5 mm from the second end, the first minimum distance between the injection site and the shortest distance to the optical area may be between 9 mm and 14 mm, and the second minimum distance between the injection site and the first end may be between 28 mm and 33 mm. The length of the tapered portion in a direction from the second end to the first end may be 5 mm and 15 mm, between 7 mm and 12 mm, or 10 mm.

The tapered portion may comprise a first tapered end and a second tapered end, wherein the first tapered end may be closest to the first end of the window part, and wherein the second tapered end may be closest to the second end of the window part. The first tapered end may comprise the largest width in the tapered portion, and the second tapered end may comprise the smallest width in the tapered portion. A ratio between the width of the first tapered end and the second tapered end may be between 0.25 and 0.4 or 0.32.

For example, a most rapid reduction of mechanical stress and birefringence in the optical area of the window part may be achieved by the tapered portion having a length in a direction between the first end and the second end of 10 mm, and having a width ratio between the first end and second end of 0.32.

The injection site may be arranged at a second minimum distance between the injection site and the first end, that may be between 83% and 95%, between 87% and 94%, or between 89% and 93%, of a third minimum distance that may be between the first end and the second end.

The third minimum distance may be the shortest distance between the first end and the second end of the window part.

Having a ratio of distances of the injection site relative to the total length of the window part from the first end to the second end, that are within the above mentioned limits (i.e. the above mentioned particular limit of ratio), allows the second molten polymer material to flow from the injection site towards the distal end (i.e. first end) of the window part, in a process, where less mechanical stress will propagate towards the first end of the window part, resulting in the formation of a part of the window part, that comprises levels of mechanical stress that result in levels of birefringence, that are below 300 nm/cm, thereby resulting in the formation of the above described optical area, that allows for an improved quality in intraoral scanning.

For example, the injection site may be located at a distance between 1 mm and 5 mm from the second end, and the third minimum distance between the first end and the second end may be between 27 mm and 37 mm.

The window part may comprise a bend or a denture, wherein a first plane containing the optical area may not coincide with a second plane containing the relief area. A first plane containing the optical area may thus have an orthogonal distance that is different from zero to a second plane containing the relief area. The first end of the window part and the second end of the window part may thus be spatially separated in a vertical direction that is orthogonal relative to a direction from the first end to the second end of the window part and that is normal to a plane containing the optical are, the relief area, or the tapered portion.

The detachable sleeve may be a part of a system comprising the detachable sleeve and the handheld intraoral scanner, wherein the detachable sleeve may be releasably attached to at least a part of the scan tip of the handheld intraoral scanner.

According to the aspect, a method for manufacturing a detachable sleeve is provided. The detachable sleeve may comprise a cover part and a window part.

The detachable sleeve may be a rigid part or a flexible part. The detachable sleeve may be a part that may be slid, clamped, screwed, or in any other way fastened into position, on the handheld intraoral scanner. The detachable sleeve may be detachably (i.e. removably) attached to the handheld intraoral scanner, such that the detachable sleeve may be removed from the handheld intraoral scanner, without the use of tools.

The handheld intraoral scanner may be configured to acquire light information reflected from a three-dimensional dental object during a scanning session, wherein the scanning session is a period of time in which a scan is performed using the handheld intraoral scanner. The light information may be intraoral scan data and may be configured to be used to generate or update a virtual 3D model of a dental object. The dental object may be a tooth, a part of a tooth, teeth, an upper or lower jaw or parts of them, a whole dentition or a part of a dentition, and/or a gingiva or part thereof. The handheld intraoral scanner may comprise a scan tip that may be arranged at a distal end of the handheld intraoral scanner, and that may be configured to be at least partly inserted inside the oral cavity (i.e. mouth) of a patient. The scan tip may comprise an open channel that may be configured to allow light to pass through two openings each located at opposite ends of the scan tip. The scan tip at the distal end may further comprise one of the two openings. The opening at the distal end of the scan tip may be covered by a cover, such as a window, that may be made of a transparent material, such as glass or a plastic material.

The detachable sleeve may be configured to cover at least a part of the scan tip. The detachable sleeve may be detachably arranged, fastened, or attached onto the scan tip of the handheld intraoral scanner.

A detachable sleeve that is configured to be removably attached to the scan tip of a handheld intraoral scanner may be beneficial, since a such detachable sleeve may improve hygiene of the equipment, the scanning process, and the patient, since a such detachable sleeve may protect the scan tip from being contaminated by coming into contact with body liquids from the patient, such as saliva and blood, thereby protecting the equipment and a next patient. The detachable sleeve may further reduce negative impact on the environment, since using single-use sleeves such as the detachable sleeve, produces less CO2 than autoclaving multiple-use sleeves, when measured during the same time period.

The detachable sleeve may comprise a cover part and a window part. The window part may be configured to be at least partly aligned with the window of the scan tip, when the detachable sleeve is arranged into position on the scan tip, and may be configured to allow light to pass to and from the window of the scan tip.

The window part may include a first end and a second end. The first end may be arranged at a distal end of the window part that is closest to a distal end of the handheld intraoral scanner that is the part that is configured to be inserted in the mouth of a patient, and the second end may be arranged at a proximal end of the window part, that is opposite to the first end, and that is closest to a housing of the handheld intraoral scanner that is configured to be held by a user of the handheld intraoral scanner.

The window part may include a tapered portion at the second end. The tapered portion may be an extension of the second end of the window part that points or tapers away from the first end of the window part or the optical area. The window part may be arranged in the detachable sleeve such, that the tapered portion tapers or points towards the opening of the detachable sleeve that may be configured to receive the at least part of the scan tip of the handheld intraoral scanner. Thus, when the detachable sleeve is attached to the scan tip of the handheld intraoral scanner and thereby covering the at least part of the scan tip of the handheld intraoral scanner, the tapered portion may taper or point away from the distal end of the scan tip of the handheld intraoral scanner. The first end of the window part may thus be arranged closer to the distal end of the scan tip of the handheld intraoral scanner than the second end of the window part is. The second end of the window part may, when attached to the scan tip of the handheld intraoral scanner, be arranged closer to a user interface (e.g. physical or digital buttons) arranged on the handheld intraoral scanner, than the first end of the window part is.

The tapered portion may allow reducing mechanical stress and resulting birefringence in other portions of the window part due to the geometry of the tapered portion.

The method may comprise manufacturing a detachable sleeve. The detachable sleeve may comprise a cover part and a window part. The method may comprise injection molding the window part. The window part may comprise a first end and a second end. The window part may comprise a tapered portion at the second end. The method may comprise manufacturing the detachable sleeve for a handheld intraoral scanner.

The method may comprise injection molding the window part. Forming the window part by injection molding may allow for a higher production rate, than forming the window using other manufacturing methods, such as forming the window in an industrial oven or other type of molding techniques.

Injection molding parts may imply injecting molten polymer material under high pressures, such as 800 bar and 1200 bar. Such high pressures may result in the formation of mechanical stress inside the material, which in turn may lead to an increase in birefringence in the material. The highest stress and highest birefringence in the injection molded part may be located closest to the injection site, which is the location in the injection molted part where the molten polymer material has entered the form that was used manufacture the injection molted part.

The window part comprising the tapered portion, may allow the injection site to be arranged at the tapered portion, and may thus allow the highest stress, and thus the highest birefringence, to be located and formed in the tapered portion, thereby relieving the rest of the window part from forming or containing the same level of stress and birefringence. The even increase in width of the tapered portion from the injection site towards the first end of the window part allows for a more uniform and laminar flow of a molten polymer material from the injection site during injection, that allows for a reduction of mechanical stress, and thereby also reduction of birefringence, forming at an area of the window part that is outside the tapered portion and that is closer to the first end of the window part than the tapered portion is, than if the width was constant through the entire length of the window part, such as in a substantially rectangular window. Furthermore, the distance from the injection site to the area of the window part outside the tapered portion allows for a further reduction of mechanical stress and birefringence at that area, such that the larger the distance, the less mechanical stress and birefringence is formed at the area of the window part outside the tapered portion. The detachable sleeve may be configured to be releasably attached to the handheld intraoral scanner such, that the polarized light to and from the handheld intraoral scanner only passes through the area of the window part that is outside the tapered portion and closer to the first end of the window part than to the second end of the window part, which is the area of the window part than comprises least mechanical stress and least birefringence, since polarized light through the window part may be refracted more in the tapered portion than in the rest of the window part that is closer to the first end of the window part than to the tapered portion, thereby allowing for an increase in scan quality.

Furthermore, since a window part comprising a tapered portion comprising the injection site allows for a more rapid reduction in mechanical stress and birefringence through the entire length of the window part in a direction away from the injection site and the tapered portion than in a window part not comprising a tapered portion comprising the injection site (e.g. a substantially rectangular or oval window), the increased reduction in mechanical stress and birefringence allows for a window part that is shorter in length between the first end and the second end, than a window part not comprising a tapered portion comprising the injection site, thereby allowing for a reduction in a length of the window part, thereby allowing for a reduction in material cost.

Since an area of the window part located at the first end, may be the area of the window part that is configured to be aligned with the opening of the scan tip, and may thus be the area of the window part that is configured to allow the light to pass to and from the handheld intraoral scanner. The area of the window part that may be configured to allow light to pass from and to the scan tip may be referred to as the “optical area”, which may be a field-of-view of the handheld intraoral scanner, which defines the area of the image data that is captured by each scan. Reducing the level of mechanical stress and birefringence within the optical area may be beneficial, since the polarized light to and from the scan tip of the handheld intraoral scanner may pass through the optical area of the window part of the detachable sleeve, and the lower the level of mechanical stress and birefringence is in the optical area, the less the polarized light may refract when passing through the optical area of the window part of the detachable sleeve.

Since the window part may comprise a tapered portion, the window part may comprise a width that smoothly increases in the tapered portion in a direction from the second end towards the first end. The smooth increase of the width in the tapered portion towards the first end of the window part may further contribute to a more rapid reduction of mechanical stress and a more rapid reduction of birefringence in the optical area of the window part, than in a window part of similar size without a smooth increase of width of a tapered portion. The tapered portion may thus extend flush from the window part. By extending flush from the window part, the tapered portion may not comprise sharp corners or sudden bends that may lead to the formation of mechanical stress during or after manufacturing the window part by injection molding the polymer material into the form, thereby reducing the formation of mechanical stress in the window part, thereby reducing birefringence in the window part.

Using the tapered portion as a relief area may relieve the formation of mechanical stress in the optical area, thereby reducing stress-induced birefringence in the optical area of the window part. For the same reason (i.e. reducing mechanical stress and birefringence), the tapered portion may not comprise concave corners. Concave corners are corners that extend inwardly towards the window part, thereby forming areas in the window part, wherein the molten polymer material will follow a rapidly expanding area in the form, during injection, which may lead to higher mechanical stress, and may thus result in higher levels of birefringence.

According to the aspect, a detachable sleeve configured to detachably cover at least a part of a handheld intraoral scanner is disclosed. The detachable sleeve may comprise a cover part, and a window part including a first end and a second end, wherein the window part may be formed by injection molding, and wherein the window part may include a tapered portion at the second end.

The method may comprise combining the cover part and the window part based on two-component injection molding.

In one aspect, the method may comprise manufacturing the cover part and the window part separately. For example, injection molding the cover part and the window part in a separate molding process. The method may comprise a step for subsequently assembling the cover part and the window, for example by gluing the cover part and the window part together.

In another aspect, the method may comprise a step of manufacturing the detachable sleeve in one piece. The cover part and the window part may be combined based on two-component injection molding, thus, the method may comprise a step of two-component injection molding the detachable sleeve by two-component injection molding the cover part and the window part.

The method may for example comprise manufacturing the detachable sleeve by injecting a first molten polymer material (e.g. Acrylonitrile Butadiene Styrene (ABS) material) into a first form, that may be shaped as the cover part of the detachable sleeve. The first form may comprise a first core for preventing the first molten polymer material to enter an opening in the formed cover part, where the window part will subsequently be located. In a subsequent step, the first core may be substituted with a second core that allows a second molten polymer material to enter the opening where the window part will be located. In another aspect of the subsequent step, the first core may be moved, such that the opening where the window part will be located is uncovered, allowing a second molten polymer material to enter the opening where the window part will be located. A second molten polymer material (e.g. Poly-methyl methacrylate material) may subsequently be injected into the opening where the window part will be located. The opening may be limited by sidewalls of the formed cover part and the substituted second core or the moved first core, thus defining a second form. The second form may thus be defined by the opening in the formed cover part and the first core that has been moved or a second core substituting the first core. The opening and the substituted second core or the moved first core may be shaped as the window part that is to be formed. The second molten polymer material may be injected at a location where the tapered portion will be formed. The second molten polymer may be injected into the second form with a pressure between 850 bar and 1150 bar. The duration of the injection may be between 150 milliseconds and 350 milliseconds.

A second pressure may subsequently be applied to the second form that may be between 850 bar and 1150 bar that may be maintained for a time period of between 350 milliseconds and 650 milliseconds.

Applying a second pressure subsequently to injecting the second molten polymer material, may allow reducing mechanical stress and resulting birefringence in the formed window part.

In another example, the detachable sleeve may be manufactured by injecting the second molten polymer material (e.g. Poly-methyl methacrylate material) into a second form shaped as the window part. The second molten polymer material may be injected at a location where the tapered portion will be formed. The second molten polymer may be injected into the second form with a pressure between 950 bar and 1050 bar. The duration of the injection may be between 210 milliseconds and 270 milliseconds.

A second pressure may subsequently be applied to the second form that may be between 850 bar and 950 bar that may be maintained for a time period of between 450 milliseconds and 550 milliseconds.

Subsequently, a first molten polymer material (e.g. Acrylonitrile Butadiene Styrene material) may be injected into a first form. The first form may be shaped as the cover part of the detachable sleeve. The first molten polymer material may be injected into the first form while the formed window part is arranged in the first form. The first form may comprise the window part while the first molten polymer material is injected into the first form, for preventing the first molten polymer material to enter a space inside the first form that is covered by the formed window part.

As described in the above examples, the detachable sleeve may be manufactured by two-component injection molding the cover part on top of the formed window part or vice versa, i.e. two-component injection molding the window part on top of the formed cover part.

The method may comprise two-component injection molding the cover part and the window part by may occur by injecting a second molten polymer material into a second form for the cover part while the window part may be arranged within the second form, or by injecting a second molten polymer material into a second form for the cover part prior to injecting the first molten polymer material into the first form for the window part while the first form may be arranged within the cover part. For example, by injecting a second molten polymer material into a second form for the cover part and subsequently injecting the first molten polymer material into the first form for the window part while the first form may be arranged within the cover part.

Two-component injection molding the detachable sleeve may reduce the production time since an assembly step, wherein the cover parts and the window parts are subsequently assembled in a separate manufacturing process, may be omitted, and may thus allow for a lower production time and a higher production rate.

Manufacturing the detachable sleeve by combining the cover part and the window part based on two-component injection molding may allow for a faster and more efficient production of detachable sleeves, since no further processing step may be required to assemble the cover part and the window part together, since one of the cover part or the window part, may be injection molded on top of the other one, and may thereby allow for gluing the cover part and window part together during one of the first or second injection molding process. The gluing may occur due to the heat generated during injection molding.

The method may comprise making the window part at least partly of a Poly-methyl methacrylate material, a Polycarbonate material, or a Cyclic Block Copolymer (e.g. poly(cyclohexylethylene) and ethylene-co-1-butene, with an adjusted ratio), and making the cover part at least partly of an Acrylonitrile Butadiene Styrene material, a Polycarbonate material, a Polybutylene terephthalate material, an Acrylonitrile butadiene styrene material, a Polypropylene material, a Polyethylene material, a Polyoxymethylene material, a Polystyrene material, or a Styrene-acrylonitrile resin material.

The method may comprise making the window part and the cover part at least partly of a Poly-methyl methacrylate material, a Polycarbonate material, or a Cyclic Block Copolymer (e.g. poly(cyclohexylethylene) and ethylene-co-1-butene, with an adjusted ratio).

The window part may be at least partly made of a Poly-methyl methacrylate material, a Polycarbonate material, or a Cyclic Block Copolymer (e.g. poly(cyclohexylethylene) and ethylene-co-1-butene, with an adjusted ratio). The cover part may be at least partly made of an Acrylonitrile Butadiene Styrene material, a Polycarbonate material, a Polybutylene terephthalate material, an Acrylonitrile butadiene styrene material, a Polypropylene material, a Polyethylene material, a Polyoxymethylene material, a Polystyrene material, or a Styrene-acrylonitrile resin material. The window part and the cover part may at least partly be made of a Poly-methyl methacrylate material, a Polycarbonate material, or a Cyclic Block Copolymer (e.g. poly(cyclohexylethylene) and ethylene-co-1-butene, with an adjusted ratio).

Poly-methyl methacrylate (PMMA) material is in particular suitable to be used as a window material, since injection molding Poly-methyl methacrylate using the above mentioned pressure ranges and pressure holding times may allow forming a window part with reduced levels of mechanical stress in the window part and reduced levels of birefringence.

The method may comprise shaping the tapered portion at least partly as one of a trapezoid, a trapezoid having a rounded end, a trapezoid having rounded corners at the second end, a triangle, a triangle having a rounded corner at the second end, semioval, a semioval tapered towards the second end, a part of a semioval, a semicircle, or a part of a semicircle.

The tapered portion may comprises rounded corners. The tapered portion may be pointed at the second end. The tapered portion may be arranged such that it extends and narrows towards the second end, and may comprise a rounded corner or a blunt portion that may comprise two rounder corners. The tapered portion may comprise rounded corners where the tapered portion meets with the rest of the window part.

The rounded corners may allow for a reduction in mechanical stress when formed and thereby, a reduction in birefringence in the formed window part is obtained, as no sharp corners are present in a flow path from the injection site towards the first end of the window part.

The tapered portion may be at least partly shaped as one of a trapezoid, a trapezoid having a rounded end, a trapezoid having rounded corners at the second end, a triangle, a triangle having a rounded corner at the second end, semioval, a semioval tapered towards the second end, a part of a semioval, a semicircle, or a part of a semicircle.

The geometry of the window part may have an effect on the birefringence of the window part, since sharp edges, concave corners, and complex geometry, may increase mechanical stress in the window part, which may increase the birefringence in the window part. Therefore, having a tapered portion in the window part that increases smooth in width from the second end towards the first end as the shapes described above, may allow for a more rapid reduction in birefringence in the optical area of the window part.

The window may be injection molded from an injection site. The tapered portion of the window part may comprise the injection site.

The injection site may be at a location at which the second molten polymer material may be injected into the second form. The highest mechanical stress and the highest birefringence in the window material may be located at the injection site. Therefore, arranging the injection site at the second end of the window part, and thus distantly from the first end of the window, at which the optical area of the window part is located, may relieve the optical area from increased mechanical stress, and may reduce mechanical stress, and thereby also reduce birefringence, in the optical area of the window part. Therefore, the injection site may be located at the tapered portion, and may preferably be located at the second end of the window part within the tapered portion.

As previously mentioned, the window part may comprise an optical area that may be a part of the window part. The optical area may be configured to allow light to pass from and to the scan tip.

Therefore, the optical area may preferably be the part of the window part that has the lowest levels of birefringence.

The optical area may be arranged closer to the first end of the window part than to the second end of the window part. Arranging the optical area closer to the first end of the window part than to the second end of the window part, may reduce the level of birefringence in the optical area, since the highest levels of birefringence may be located closest to the injection site, which may be located at the second end of the window part. This may be because the tapered portion allows for a more rapid decrease of a level of birefringence in a direction from the injection site towards the first end of the window part, and therefore, a minimal birefringence is achieved at the optical area, as the optical area is arranged furthest away from the injection site. The optical area may comprise levels of birefringence between 50 nm/cm and 350 nm/cm, such as for example between 100 nm/cm and 300 nm/cm. The optical area of the window part may be defined as an area, region, or part of the window part, that has a level of birefringence between 100 nm/cm and 300 nm/cm.

Reducing the level of mechanical stress and birefringence within the optical area may improve the quality of intraoral scanning, since the polarized light to and from the scan tip of the handheld intraoral scanner performing the scanning, may pass through the optical area of the window part of the detachable sleeve covering the scan tip. The lower the level of mechanical stress and birefringence is in the optical area, the less the polarized light may refract when passing through the optical area of the window part of the detachable sleeve, and thus the higher the quality of the intraoral scanning may be.

The window part may comprise a relief area arranged at the second end of the window part. The relief area may be a part of the window part that is configured to contain the highest levels of mechanical stress and the highest level of birefringence, thereby relieving the optical area of the window part from containing the highest levels of mechanical stress and the highest levels of birefringence. Concentrating the highest levels of mechanical stress and the highest levels of birefringence in the relief area may be achieved by arranging the injection site, within the relief area. For example, the injection site or the relief area may be arranged as far from the optical areas as possible. Arranging the injection site as far as possible from the optical area may be achieved by injecting the second molten polymer material at a location at the second form where the second end of the window part will be located. The relief area may be within the tapered portion of the window part, and may be an area of the tapered portion at which the injection site is located.

Concentrating the highest levels of mechanical stress and highest levels of birefringence in the relief area may allow the optical area to have reduced levels of mechanical stress and reduced levels of birefringence, thereby improving the quality of the intraoral scanning.

For example, the window part may have a length of 300 mm from the first end to the second end, the optical area may be located between the first end of the window part and a distance of 150 mm from the first end. The relief area may be located between the second end of the window part and 150 mm from the second end. The window part may be divided between the optical area and the relief area.

A transition area may be located between the optical area and the relief area of the window part. The transition area may be a part of the window part that comprises levels of mechanical stress and levels of birefringence that are higher than levels of mechanical stress and levels of birefringence of the optical area, but lower than the levels of mechanical stress and levels of birefringence of the relief area. The optical area may be located between the first end of the window part and the transition area. The relief area may be located between the second end of the window part and the transition area.

A ratio between a first minimum distance between the injection site and the optical area and a second minimum distance between the injection site and the first end of the window part may be between 25% and 45%. The ratio may be between 33% and 43%. The ratio may be between 36% and 40%.

The first minimum distance may be a first distance between the injection site and the closest part of the optical area to the injection site.

The second minimum distance may be a second distance between the injection site and the closest part of the first end of the window part.

Having a ratio of distances of the injection site relative to the optical area and a distance between the injection site and the distal end of the window part that are within the above mentioned limits (i.e. the above mentioned particular limit of ratio), allows the second molten polymer material to flow from the injection site towards the distal end (i.e. first end) of the window part, in a process, where less mechanical stress will propagate towards the first end of the window part, resulting in the formation of a part of the window part, that comprises levels of birefringence, that are below 300 nm/cm, thereby resulting in the formation of the above described optical area, that allows for an improved quality in intraoral scanning.

For example, the injection site may be located at a distance between 1 mm and 5 mm from the second end, the first minimum distance between the injection site and the shortest distance to the optical area may be between 9 mm and 14 mm, and the second minimum distance between the injection site and the first end may be between 28 mm and 33 mm. The length of the tapered portion in a direction from the second end to the first end may be 5 mm and 15 mm, between 7 mm and 12 mm, or 10 mm.

The tapered portion may comprise a first tapered end and a second tapered end, wherein the first tapered end may be closest to the first end of the window part, and wherein the second tapered end may be closest to the second end of the window part. The first tapered end may comprise the largest width in the tapered portion, and the second tapered end may comprise the smallest width in the tapered portion. A ratio between the width of the first tapered end and the second tapered end may be between 0.25 and 0.4 or 0.32.

For example, a most rapid reduction of mechanical stress and birefringence in the optical area of the window part may be achieved by the tapered portion having a length in a direction between the first end and the second end of 10 mm, and having a width ratio between the first end and second end of 0.32.

The method may comprise injecting the first molten polymer material at an injection site at a location in the first form where an injection site may be located in the tapered portion of the formed window part. The window part may comprise an injection site at which the first molten polymer material was injected into the first form. The injection site may be arranged at a second minimum distance between the injection site and the first end, that is between 83% and 95%, between 87% and 94%, or between 89% and 93%, of a third minimum distance between the first end and the second end.

The injection site may be arranged at a second minimum distance between the injection site and the first end, that may be between 83% and 95%, between 87% and 94%, or between 89% and 93%, of a third minimum distance that may be between the first end and the second end.

The third minimum distance may be the shortest distance between the first end and the second end of the window part.

Having a ratio of distances of the injection site relative to the total length of the window part from the first end to the second end, that are within the above mentioned limits (i.e. the above mentioned particular limit of ratio), allows the second molten polymer material to flow from the injection site towards the distal end (i.e. first end) of the window part, in a process, where less mechanical stress will propagate towards the first end of the window part, resulting in the formation of a part of the window part, that comprises levels birefringence, that are below 300 nm/cm, thereby resulting in the formation of the above described optical area, that allows for an improved quality in intraoral scanning.

For example, the injection site may be located at a distance between 1 mm and 5 mm from the second end, and the third minimum distance between the first end and the second end may be between 27 mm and 37 mm.

The method may comprise injecting a first molten polymer material into a first form for the window part at a first pressure of between 850 bar and 1150 bar, between 900 bar and 1100 bar, or between 950 bar and 1050 bar. The injection may occur at a speed between 150 milliseconds and 350 milliseconds, between 200 milliseconds and 300 milliseconds, or between 210 milliseconds and 270 milliseconds.

The method may comprise maintaining a second pressure of between 850 bar and 1150 bar, between 900 bar and 1100 bar, or between 950 bar and 1050 bar. The method may comprise maintaining the second pressure for a time period of between 350 milliseconds and 650 milliseconds, between 400 milliseconds and 600 milliseconds, or between 450 milliseconds and 550 milliseconds. The second pressure may be applied subsequent to the first pressure.

In another example, the method may comprise maintaining a second pressure of 900 bar for a time period of 0.5 seconds. The second pressure may be applied subsequent to the first pressure.

For example, the method may comprise injecting a first molten polymer material (e.g. Acrylonitrile Butadiene Styrene material) into a first form, that may be shaped as the cover part of the detachable sleeve. The first form may comprise a first core for preventing the first molten polymer material to enter an opening in the formed cover part, where the window part will subsequently be located. In a subsequent step, the first core may be substituted with a second core that allows a second molten polymer material to enter the opening where the window part will be located. In another aspect of the subsequent step, the first core may be moved such, that the opening where the window part will be located is uncovered, allowing a second molten polymer material to enter the opening where the window part will be located.

A second molten polymer material (e.g. Poly-methyl methacrylate material) may subsequently be injected into the opening where the window part will be located. The opening may be limited by sidewalls of the formed cover part and the substituted second core or the moved first core. The opening and the substituted second core or the moved first core may be shaped as the window part that is to be formed. The second molten polymer material may be injected at a location where the tapered portion will be formed. The second molten polymer may be injected into the second form with a pressure between 850 bar and 1150 bar. The duration of the injection may be between 150 milliseconds and 350 milliseconds.

A second pressure may subsequently be applied to the second form that may be between 850 bar and 1150 bar that may be maintained for a time period of between 350 milliseconds and 650 milliseconds.

Applying a second pressure subsequently to injecting the second molten polymer material, may allow reducing mechanical stress and resulting birefringence in the formed window part.

The window part may comprise a bend or a denture, wherein a first plane containing the optical area may not coincide with a second plane containing the relief area. A first plane containing the optical area may thus have an orthogonal distance that is different from zero to a second plane containing the relief area. The first end of the window part and the second end of the window part may thus be spatially separated in a vertical direction that is orthogonal relative to a direction from the first end to the second end of the window part and that is normal to a plane containing the optical are, the relief area, or the tapered portion.

The detachable sleeve may be a part of a system comprising the detachable sleeve and the handheld intraoral scanner, wherein the detachable sleeve may be releasably attached to at least a part of the scan tip of the handheld intraoral scanner.

Those skilled in the art will recognize still other aspects of the present application upon reading and understanding the attached description.

### BRIEF DESCRIPTION OF THE DRAWINGS

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 schematically illustrates a handheld intraoral scanner system comprising a handheld intraoral scanner and a detachable sleeve;

FIGS. 2A-2B illustrate the detachable sleeve seen from a front and a side view, respectively;

FIG. 3 illustrates the window part of the detachable sleeve;

FIGS. 4A-4E schematically illustrate exemplary shapes of the window part of FIG. 3;

FIG. 5 schematically illustrates an exemplary setup for injection molding a window part of the detachable sleeve;

FIG. 6 schematically illustrates an exemplary setup for two-component injection molding the detachable sleeve in two steps; and

FIGS. 7A, 7B. and 7C illustrate different examples of a table with an overview of a method for injection molding a window part of the detachable sleeve.

### DETAILED DESCRIPTION OF THE DRAWINGS

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

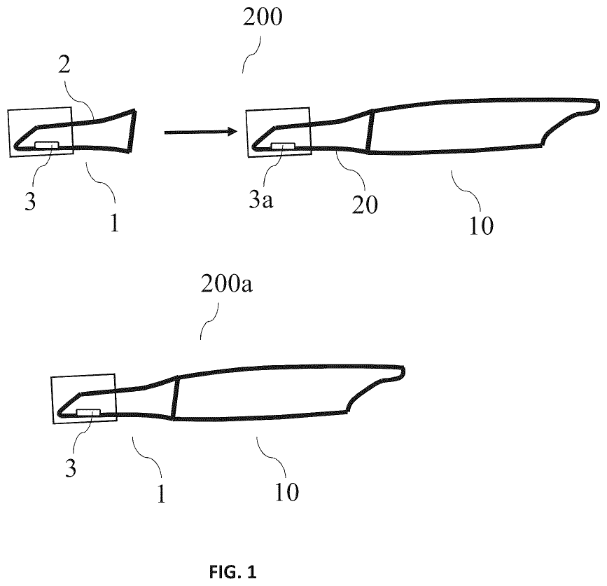

FIG. 1 illustrates a handheld intraoral scanner system 200 comprising a handheld intraoral scanner 10 and a detachable sleeve 1. The handheld intraoral scanner 10 comprises a scan tip 20 that is configured to direct light from a light source inside the handheld intraoral scanner 10 through a transparent cover 3a (e.g. a window) of the scan tip towards a dental object inside a patient's mouth, and direct the reflected light from the dental object through the transparent cover 3a of the scan tip and towards a photosensor inside the handheld intraoral scanner 10. The scan tip 20 is configured to be partly inserted into the mouth of a patient. A detachable sleeve 1 is seen comprising a cover part 2 and a window part 3. The detachable sleeve 1 may be configured to be detachably attached to the scan tip 20 of the handheld intraoral scanner 10. The detachable sleeve 1 may be configured to be releasably attached to the scan tip 20 without the need of tools, and my be attached and released by a clamping function, by friction, or be screwing it to the scan tip 20 or to another part of the handheld intraoral scanner 10. The detachable sleeve 1 may be configured to protect the scan tip 20 from becoming contaminated by body liquids from the mouth of a patient, such as saliva or blood, and may become disposed after use. The detachable sleeve 1 may be hollow and configured to accommodate at least a part of the scan tip 20 of the handheld intraoral scanner 10. An arrow indicates the direction of insertion of the detachable sleeve 1 onto the scan tip 20. The cover part 2 may be made of a plastic material such as Acrylonitrile Butadiene Styrene. The window part 3 may be made of a plastic material such as Poly-methyl methacrylate. The window part 3 may be configured to be aligned with the transparent cover 3a of the scan tip 20, such that the light that passes through the transparent cover 3a also passes through the window part 3.

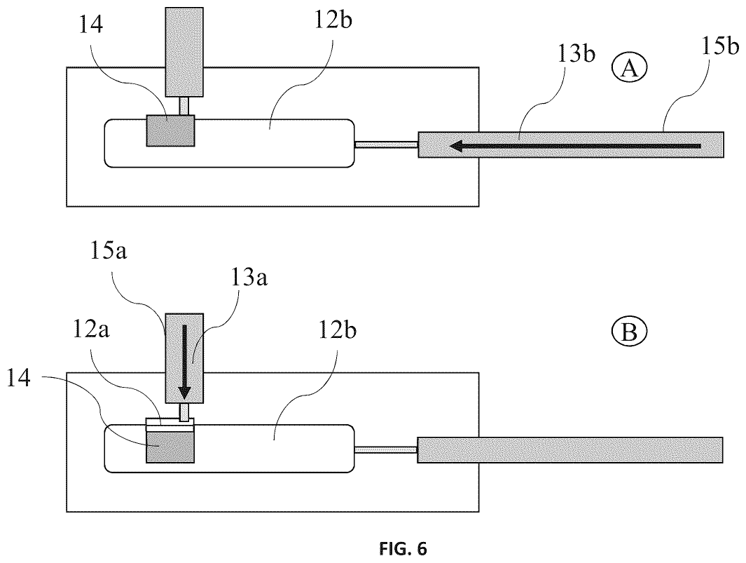

The window part 3 may be formed by injection molding, and subsequently attached to the cover part 2 by for example gluing. Alternatively, the window part 3 may be formed by a two-component injection molding process, wherein the window part 3 is injection molded into a first form 12a (shown in FIG. 6A) at least partly made of the formed cover part 2 subsequently to the injection molding of the cover part 2, or wherein the window part 3 is injection molded into a first form 12a and wherein the cover part 2 is subsequently injection molded into a second form 12b (shown in FIG. 6A) that comprises the formed window part 3. FIG. 1 further shows a handheld intraoral scanner system 200a wherein the detachable sleeve 1 is attached to the handheld intraoral scanner 10.

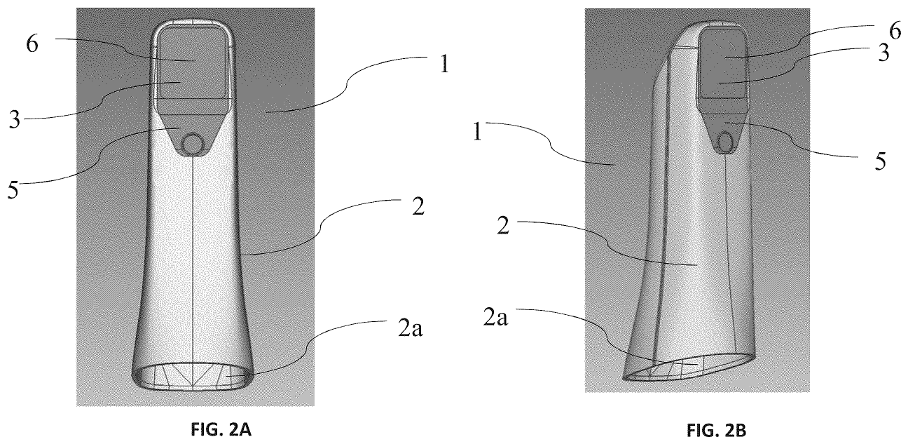

FIGS. 2A-2B illustrate the detachable sleeve 1 seen from a front and a side view, respectively.

FIG. 2A illustrates the detachable sleeve 1 from a front view. The figure shows the window part 3 attached to the cover part 2. The figure further shows an open channel 2a through the cover part 2. The open channel 2a is configured to accommodate the scan tip 20 of the handheld intraoral scanner 1. The figure further shows an optical area 6 of the window part 3. The optical area may be a part of the window part 3 that is aligned with the transparent cover 3a of the scan tip 20, when the detachable sleeve 1 is attached to the scan tip 20. The optical area 6 may therefore be the part of the window part 3 that has the least levels of mechanical stress and thus least levels of birefringence. The figure further shows a tapered portion 5 of the window part 3. The tapered portion 5 may have a trapezoidal shape.

FIG. 2B illustrates the detachable sleeve 1 of FIG. 2A from a side view. The window part 3 may have a thickness of 4 mm or less, such as between 0.4 mm and 4 mm.

From this figure it is further seen that the window part 3 comprises a bend or a denture between the tapered portion 5 and the optical area 6, wherein a first plane containing the optical area 6 is not coincide with a second plane containing the relief area 7. A first plane containing the optical area 6 has thus an orthogonal distance that is different from zero to a second plane containing the relief area 7. The first end 4a of the window part 3 and the second end 4b of the window part 3 are thus spatially separated in a vertical direction that is orthogonal relative to a direction from the first end 4a to the second end 4b of the window part 3 and that is normal to a plane containing the optical are 6, the relief area 7, or the tapered portion 5.

The detachable sleeve 3 is seen configured to be releasably attached to the handheld intraoral scanner 10 such, that the polarized light to and from the handheld intraoral scanner 10 only passes through the area of the window part 3 that is outside the tapered portion 5 and closer to the first end 4a of the window part 3 than to the second end 4b of the window part 3, which is the area of the window part 3 that comprises least mechanical stress and least birefringence. The optical area 6 is the field-of-view of the handheld intraoral scanner 10, which defines the area of the image data that is captured by each scan. Thus, the optical area 6 is the field-of-view of the handheld intraoral scanner 10.

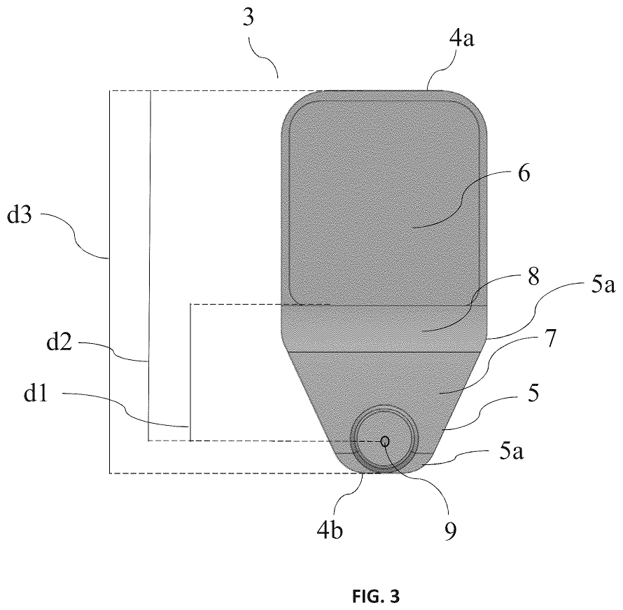

FIG. 3 illustrates one example of a shape of the window part 3 of the detachable sleeve 1. The figure shows the window part 3 comprising a first end 4a, a second end 4b, the optical area 6 located closer to the first end 4a than to the second end 4b, and the tapered portion 5 located closer to the second end 4b than to the first end 4a. The figure further shows an injection site 9 located at the tapered portion 5. The injection site 9 may be a location at which a first molten polymer material was injected into the first form 12a during the forming of the window part 3. The tapered portion 5 may increase in width towards the first end of the window part 3 (i.e. towards the optical area 6), and decrease in width towards the second end 4b. The injection site 9 is shown located at the tapered portion 5 closer to the second end 4b than to the optical area 6. Since the width of the tapered portion increases towards the first end 4a of the window part, and the injection site 9 is located closer to the second end 4b than to the optical area 6, the tapered portion may provide a stress relieving function for the optical area, thus defining a relief area 7, relieving the optical area 6 of some mechanical stress, thus allowing for reduced levels of birefringence at the optical area 6.

The increasing width of the tapered portion 5 allows the first molten polymer material to flow from the injection site 9 during injection such, that less mechanical stress, and thereby also less birefringence, will form at the optical area 6 of the window part 3, than if the width was constant through the length of the entire window part 3. Furthermore, the distance from the injection site 9 to the optical area 6 further allows for a reduction of mechanical stress and birefringence at the optical area 6, such that the larger the distance, the less mechanical stress and birefringence is formed at the optical area 6.

The figure thus further shows a relief area 7 of the window part 3, that may comprise the injection site 9. The relief area 7 may be configured to relieve the optical area 6 from mechanical stress, and thus may be the part of the window part 3 that has the highest levels of mechanical stress and thus highest levels of birefringence.

The figure further shows a transition area 8 located intermediate between the optical area 6 and the relief area 7. The figure further shows rounded corners 5a at second end 4b of the window part 3 and at the transition area 8 where the width of the window part 3 begins to decrease. The rounded corners 5a may further contribute to the relieving function of the tapered portion 5 of the window part 3.

FIG. 3 further shows a first minimum distance d1 between the injection site 9 and the optical area 6, which is the shortest distance between the injection site 9 and the optical area 6 that is closest to the injection site 9.

The figure further shows a second minimum distance d2 between the injection site 9 and the first end 4a, which is the shortest distance between the injection site 9 and the first end 4a that is closest to the injection site 9.

The figure further shows a third minimum distance d3 between the second end 4b and the first end 4a, which is the shortest distance between the second end 4b and the first end 4a.

In a first example, the injection site may be located 3 mm from the second end 4b, d1 may be 11.5 mm, d2 may be 30 mm, and d3 may be 33 mm.

In a second example, the injection site may be located between 1 mm and 6 mm, d1 may be between 9 mm and 14 mm, d2 may be between 27 mm and 33 mm, and d 3 may be between 27 mm and 37 mm.

The tapered portion 5 of the window part 3 is seen arranged on the detachable sleeve 1 such, that the tapered portion 5 tapers or points towards an opening of the detachable sleeve 1 that is configured to receive the at least part of the scan tip 20 of the handheld intraoral scanner 10.

The first end 4a of the window part 3 is seen arranged closer to a distal end of the scan tip 20 of the handheld intraoral scanner 10 than the second end 4b of the window part 3 is. The second end 4a of the window part 3 is seen arranged opposite to the first end 4a of the window part 3.

The tapered portion 5 is seen extending and narrowing towards the second end 4b and seen comprising two rounded corners 5a at the second end 4b.

The tapered portion 5 is seen comprise a first tapered end and a second tapered end, wherein the first tapered end is closest to the first end 4a of the window part 3, and wherein the second tapered end is closest to the second end 4b of the window part 3. The first tapered end comprises the largest width in the tapered portion 5, and the second tapered end comprises the smallest width in the tapered portion 5. A ratio between the width of the first tapered end and the second tapered end is anticipated to be between 0.25 and 0.4 or 0.32.

It is further seen that no sharp corners are present in a flow path from the injection site 9 towards the first end 4a of the window part 3.

An even increase in width of the tapered portion 5 from the injection site 9 towards the first end 4a of the window part 3 is seen and the formation at an area of the window part 3 that is outside the tapered portion 5 and that is closer to the first end 4a of the window part 3 than the tapered portion 5 is indicated.

The injection site 9 or the relief area 7 is seen arranged as far from the optical area 6 as possible.

The window part 3 is seen including a first end 4a and a second end 4b. The first end 4a is arranged at a distal end of the window part 3 that is closest to a distal end of the handheld intraoral scanner 10 that is the part that is configured to be inserted in the mouth of a patient, and the second end 4b is seen arranged at a proximal end of the window part 3, that is opposite to the first end 4a, and that is closest to a housing of the handheld intraoral scanner 10 that is configured to be held by a user of the handheld intraoral scanner 10.

The window part 3 is seen including a tapered portion 5 at the second end 4a. The tapered portion 5 is an extension of the second end 4a of the window part 3 that points or tapers away from the first end 4a of the window part 3 or the optical area 6. The window part 3 is arranged in the detachable sleeve 1 such, that the tapered portion 5 tapers or points towards the opening of the detachable sleeve 1 that is configured to receive the at least part of the scan tip 20 of the handheld intraoral scanner 10. Thus, when the detachable sleeve 1 is attached to the scan tip 20 of the handheld intraoral scanner 10 and thereby covering the at least part of the scan tip 20 of the handheld intraoral scanner 10, the tapered portion 5 tapers or points away from the distal end of the scan tip 20 of the handheld intraoral scanner 10. The first end 4a of the window part 3 is thus arranged closer to the distal end of the scan tip 20 of the handheld intraoral scanner 10 than the second end 4b of the window part 3 is. The second end 4b of the window part 3 is, when attached to the scan tip 20 of the handheld intraoral scanner 10, arranged closer to a user interface (e.g. physical or digital buttons) arranged on the handheld intraoral scanner 10, than the first end 4a of the window part 3 is.

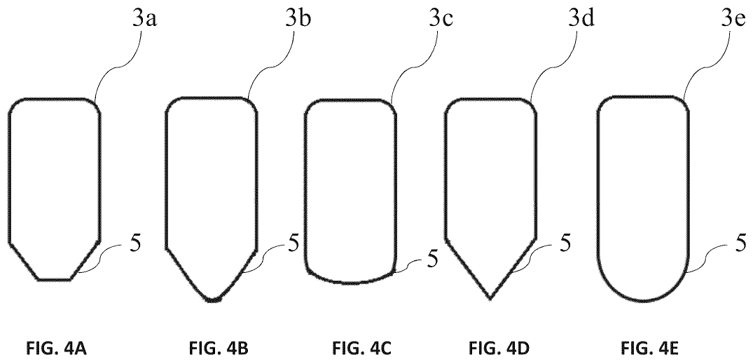

FIGS. 4A-4E schematically illustrates exemplary shapes 3a-3e of the window part 3. The figure shows that a common feature of all shown shapes 3a-3e of the window part 3 is a tapered portion 5, that is a part of the window part 3 that is located closer to the second end 4b that reduces evenly in width towards the second end 4b. The tapered portion 5 contributes to the reduction of birefringence at the portion of the window part 3 that is located closer to the first end 4a than to the second end 4b, thereby defining an optical area 6.

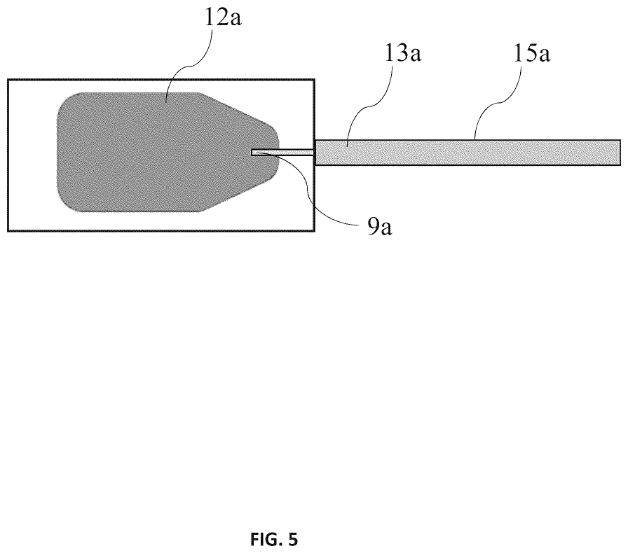

FIG. 5 schematically illustrates an exemplary setup for injection molding a window part 3 of the detachable sleeve 1. The figure illustrates a first form 12a shaped as the window part 3 of the detachable sleeve 1. A such first form 12a may be made of a metal, such as steel, and may be enclosed by an enclosure defining a mold cavity, to prevent a molten polymer to exit when injected into the form under pressure. Usually, injection molding occurs by heating a polymer material inside an injection molding machine and injected under high pressure through an injection nozzle of the injection machine into the mold cavity comprising the form. The figure shows the first molten polymer material 13a inside a first injection nozzle 15a that is set up to inject the first polymer material 13a into the first form 12a at the location of the injection site 9a.

FIG. 6 schematically illustrates an exemplary setup for two-component injection molding the detachable sleeve in two steps. In two-component injection molding a single part is formed by attaching two separate parts together, by injection molding one of the two parts onto the other of the two parts, thereby forming a single unit.

The figure schematically illustrates a first example showing a first situation A and a second situation B. The figure shows a first situation A wherein a second molten material 13b (e.g. molten Acrylonitrile Butadiene Styrene) is injected into a second form 12b through a second injection nozzle 15b of an injection molding apparatus. The second form 12b forms a second injection cavity configured to form a cover part 2 of the detachable sleeve 1 comprising an open channel 2a through the cover part 2. A core 14 is inserted into the second form 12b to cover a space in the second form 12b to keep the space from being covered by the second molten polymer material 13b when injected into the second form 12b. The space that will not be covered by the second molten polymer material 13b will subsequently in a following step be filled with the first molten polymer material 13a to form the window part 3 of the detachable sleeve 1. When the second molten polymer material 13b is injected into the second form 12b and subsequently cooled down, a solid cover part 2 of the detachable sleeve is formed.

In a subsequent step, illustrated by a second situation B, the core 14 is moved such, that the core 14 uncovers a part of the space the core 14 previously covered, and such that the core 14 in combination with the formed cover part 2, forms a first form 12a shaped as the window part 3. A first molten polymer material 13a (e.g. molten Poly-methyl methacrylate) may subsequently be injected into the first form 12a through a first injection nozzle 15a. The material of the formed cover part 2 that comes into contact with the injected first molten polymer material 13a may be heated by the heat of the first molten polymer material 13a, and thereby glued together with the formed window part 3. Subsequently, when the cover part 2 and the window part 3 are cooled down and reached a solid state, the cover part 2 and the window part 3 will constitute a single unit of a detachable sleeve 1. The injection of the first molten polymer material 13a is seen performed as distant from the optical area 9 as possible.

In a second example of a second setup for two-component injection molding the detachable sleeve 1 in two steps, the window part 3 may be formed prior to the cover part 2. In this second example, the first molten polymer material 13a (e.g. molten Poly-methyl methacrylate) may be injected through a first injection nozzle 15a into a first form 12a shaped as the window part 3 of the detachable sleeve. The first form 12a may be arranged in a second form 12b shaped as the cover part 2 of the detachable sleeve 1 and may be defined by the second form 12b and a core 14 that is further arranged in the second form 12b, as illustrated in situation B of FIG. 6. After the formed window part 3 is formed and cooled down to and reached a solid state, the second molten polymer material 13b (e.g. molten Acrylonitrile Butadiene Styrene) is injected into the second form 12b through a second injection nozzle (15b), while the formed window part 3 is still arranged in the second form 12b, that is shaped as the cover part 2 of the detachable sleeve 1, as illustrated in situation A of FIG. 6, but without the core 14, since the formed window part 3 already is arranged in the space that was covered by the core 14 in the first example. FIG. 6 further illustrates two-component injection molding by injection molding the window part 3 in a separate molding process and the cover part 2 in another separate molding process.

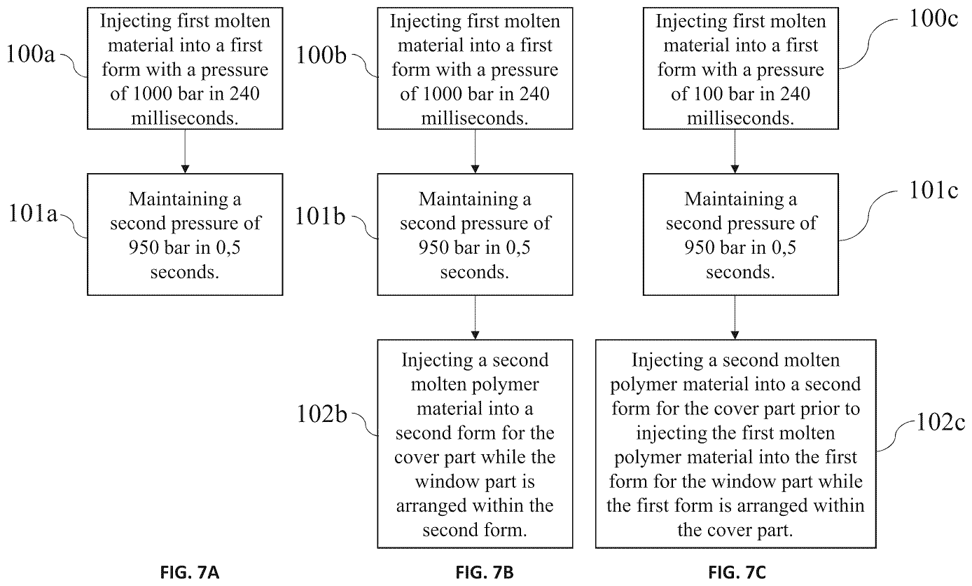

FIGS. 7A, 7B, and 7C illustrate different examples of a table with an overview of a method for injection molding a window part 3 of the detachable sleeve 1.

FIG. 7A shows a table with an overview of a method for injection molding a window part 3 of the detachable sleeve 1. In a first step 100a, a first molten material 13a is injected into a first form 12a with a pressure of 1000 bar in 240 milliseconds.

In a subsequent second step 101a, a second pressure is applied and maintained to the first form 12a at 950 bar for 0.5 seconds.

The method allows forming a window part 3 comprising a region (optical area 6) that has a reduced level of mechanical stress, and thus a reduced level of birefringence.

The formed window part 3 may subsequently be attached to a cover part 2 by way of gluing, screwing, clicking, or otherwise attaching, thereby manufacturing a detachable sleeve 1 for a handheld intraoral scanner 10.

FIG. 7B shows a table with an overview of a method for two-component injection molding a detachable sleeve, wherein the window part is formed first.

In a first step 100b, a first molten material 13a is injected into a first form 12a with a pressure of 1000 bar in 240 milliseconds.

In a subsequent second step 101b, a second pressure is applied and maintained to the first form 12a at 950 bar for 0.5 seconds.

In a third step 102b, a second molten polymer material 13b is injected into a second form 12b for the cover part 2 while the window part 3 is arranged within the second form 12b.

FIG. 7C shows a table with an overview of a method for two-component injection molding a detachable sleeve, wherein the cover part is formed first.

In a first step 100c, a first molten material 13a is injected into a first form 12a with a pressure of 1000 bar in 240 milliseconds.

In a subsequent second step 101c, a second pressure is applied and maintained to the first form 12a at 950 bar for 0.5 seconds.

In a third step 102c, a second molten polymer material 13b is injected into a second form 12b for the cover part 2 prior to injecting the first molten polymer material 13a into the first form 12a for the window part 3 while the first form 12a is arranged within the cover part 2.

FIG. 7C and FIG. 7C show tables with an overview of two methods for two-component injection molding the detachable sleeve 1 comprising a window part 3 that comprises a region (optical area 6) that has a reduced level of mechanical stress, and thus a reduced level of birefringence. The cover part 2 and the window part 3 may be attached together by the one of the two parts partly heating the other of the two parts up while being injected into its respective form, thereby allowing for an adhesion by gluing, thereby forming the detachable sleeve 1 as a single unit.

FIGS. 7B-7C schematically illustrate tables with an overview of the method of two-component injection molding the window part 3 in a separate molding process and the cover part 2 in another separate molding process.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to a component/unit/element in the singular is not intended to mean “one and only one” unless explicitly so stated, but rather “one or more.” A claim may refer to any of the preceding claims, and “any” is understood to mean “any one or more” of the preceding claims.

It is intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e., to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element, but an intervening element may also be present, unless expressly stated otherwise.

Furthermore, “connected” or “coupled” as used herein may include wirelessly connected or coupled. As used herein, the term “and/or” includes any and all combinations of one or more of the associated listed items. The step of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

1. A detachable sleeve configured to detachably cover at least a part of a handheld intraoral scanner, the detachable sleeve comprising:
a cover part; and
a window part including a first end and a second end;
wherein the window part is formed by injection molding, and
wherein the window part includes a tapered portion at the second end.

2. The detachable sleeve according to claim 1, wherein the tapered portion extends flush from the window part and wherein the tapered portion does not comprise concave corners.

3. The detachable sleeve according to claim 1, wherein the cover part and the window part are combined based on two-component injection molding.

4. The detachable sleeve according to claim 1, wherein the window part is injection molded from an injection site, and wherein the tapered portion comprises the injection site.

5. The detachable sleeve according to claim 1, wherein the window part comprises an optical area arranged at the first end of the window part, and a relief area arranged at the second end of the window part.

6. The detachable sleeve according to claim 5, wherein a ratio between a first minimum distance between the injection site and the optical area and a second minimum distance between the injection site and the first end of the window part is between 25% and 45%.

7. The detachable sleeve according to claim 4, wherein the injection site is arranged at a second minimum distance between the injection site and the first end, that is between 83% and 95% of a third minimum distance between the first end and the second end.

8. A system comprising a handheld intraoral scanner and the detachable sleeve according to claim 1.

9. A method for manufacturing a detachable sleeve, the detachable sleeve comprising a cover part and a window part, wherein the method comprising injection molding the window part, wherein the window part comprising a first end and a second end, and wherein the window part includes a tapered portion at the second end.

10. The method according to claim 9, wherein the tapered portion extends flush from the window part and wherein the tapered portion does not comprise concave corners.

11. The method according to claim 9, wherein the method comprises combining the cover part and the window part based on two-component injection molding.

12. The method according to claim 9, wherein the method comprising:
injecting a first molten polymer material into a first form for the window part at a first pressure of between 850 bar and 1150 bar at an injection speed between 150 milliseconds and 350 milliseconds.

13. The method according to claim 12, wherein the method comprising:
maintaining a second pressure of between 800 bar and 1150 bar for a time period of between 350 milliseconds and 650 milliseconds wherein the second pressure is applied subsequent to the first pressure.

14. The method according to claim 12, the method further comprising:
two-component injection molding the cover part and the window part by:
injecting a second molten polymer material into a second form for the cover part while the window part is arranged within the second form; or
injecting a second molten polymer material into a second form for the cover part prior to injecting the first molten polymer material into the first form for the window part while the first form is arranged within the cover part.

15. The method according to claim 12, wherein the window part comprises an injection site at which the first molten polymer material was injected into the first form, and wherein the injection site is arranged at a second minimum distance between the injection site and the first end, that is between 83% and 95%, of a third minimum distance between the first end and the second end.
